(19) United States
(12) Patent Application Publication
(10) Pub. No.: US 2026/0115198 A1
(43) Pub. Date: Apr. 30, 2026

(54) USE OF SRT2104 IN PREPARATION OF DRUG FOR TREATMENT OF BRONCHIAL ASTHMA

(71) Applicant: Shunde Women and Children's Hospital of Guangdong Medical University, Foshan (CN)

(72) Inventors: Yajun Wang, Foshan (CN); Guoda Ma, Foshan (CN); Junhao Guo, Foshan (CN); Riling Chen, Foshan (CN); Ning Chang, Foshan (CN); Yisen Cheng, Foshan (CN); Zhengqiang Luo, Foshan (CN); Xusan Xu, Foshan (CN); Fei Luo, Foshan (CN); Rabia Parveen, Foshan (CN); Muhammad Wasim, Foshan (CN); Keyan Lu, Foshan (CN); Houlang Wen, Foshan (CN); Guochun Huang, Foshan (CN); Yuejian Zhang, Foshan (CN); Guining Lu, Foshan (CN); Guanbang Hong, Foshan (CN); Shengxiang Fu, Foshan (CN)

(21) Appl. No.: 19/302,124

(22) Filed: Aug. 18, 2025

(30) Foreign Application Priority Data
Oct. 29, 2024 (CN) 2024115165674

(51) Int. Cl. A61K 31/5377; A61K 9/00; A61P 11/06

(52) U.S. Cl. CPC A61K 31/5377; A61K 9/0078; A61P 11/06

(57) ABSTRACT

A use of SRT2104 in preparation of a drug for treatment of bronchial asthma is provided, relating to the field of bronchial asthma treatment. The comprehensive analysis of the activation effect of SRT2104 on silent information regulator 1 (SIRT1) in the airway epithelium under asthma conditions and its alleviation effect on asthma airway inflammation is conducted. It is demonstrated that SRT2104 can be activated in the airway epithelium under asthma condition and effectively control asthma airway inflammation. A foundation is laid for the discovery of clinical drug targets and the development of drugs for the treatment of bronchial asthma.

### CROSS-REFERENCE TO RELATED APPLICATION

This application claims priority to Chinese patent application No. CN 202411516567.4, filed with China National Intellectual Property Administration (CNIPA) on Oct. 29, 2024, which is herein incorporated by reference in its entirety.

### TECHNICAL FIELD

The disclosure relates to the technical field of treatment for bronchial asthma, and more particularly to a use of SRT2104 in preparation of a drug for treatment of bronchial asthma.

### BACKGROUND

Bronchial asthma (hereinafter referred to as asthma) is recognized as one of the most common respiratory diseases. It is a heterogeneous disease characterized by chronic airway inflammation and airway remodeling, with various respiratory symptoms accompanied by reversible expiratory airflow limitation. Clinical manifestations, including wheezing, shortness of breath, chest tightness, and cough, are observed in patients.

Airway epithelial cells are regarded as playing a central role in the pathogenesis of asthma and positioned at the interface between the body and the external environment, serving as the first line of defense against microorganisms, harmful gases, and allergens. During the development of asthma, airway epithelium is repeatedly stimulated by intrinsic and extrinsic factors (biological, physical, chemical, and allergic reactions, etc.), leading to epithelial damage. Once damaged, airway epithelium allows allergens from the external environment to easily penetrate into the submucosa and activate the immune system, triggering allergic reactions. An increasing number of studies have found that the barrier function of airway epithelial cells is primarily maintained through intercellular junction mechanisms (e.g., tight junctions, adherens junctions, etc.) to prevent the entry of harmful stimuli, and through the mucociliary system and antimicrobial peptides to clear allergens, viruses, and other harmful external factors. In healthy individuals, airway epithelial cells maintain the dynamic balance of the airway environment through a biochemical barrier. However, when the airway barrier is impaired, airway structure and related proteins are damaged, leading to the onset and progression of asthma. Fungi and pollen extracts are known to increase the permeability of the epithelial barrier by affecting the expression of junctional proteins. House dust mites are reported to disrupt the barrier function of airway epithelial cells by activating protease-activated receptor 2 (PAR-2) in these cells. Furthermore, studies have shown that the impact of viruses on the barrier function of airway epithelial cells is even more significant. Infected cells undergo apoptosis or necrosis under viral induction, resulting in epithelial barrier damage. Many years ago, researchers noticed airway epithelial cell clumps in the sputum of asthma patients, and the number of such epithelial cells was found to increase significantly during exacerbations. Therefore, airway epithelial cell injury is considered a pathological feature of bronchial asthma and is closely associated with airway reactivity and disease severity. Silent information regulator 1 (SIRT1), the most extensively studied member of the Sirtuins family, is capable of sensing changes in cellular energy metabolism via nicotinamide adenine dinucleotide (NAD⁺) and modulating the acetylation levels of substrates through its deacetylase activity, thereby converting metabolic signals into epigenetic signals. Currently, it is known that SIRT1 can regulate more than 70 substrates, including p53, peroxisome proliferator-activated receptor gamma coactivator 1-alpha (PGC-1α), nuclear factor kappa-light-chain-enhancer of activated B cells (NF-κB), forkhead box O (FOXO), etc. Through interactions with different substrates, SIRT1 performs diverse functions. Thus, SIRT1 is involved in the regulation of various cellular functions and physiological and pathological processes, such as cell injury repair, growth, proliferation, apoptosis, autophagy, and differentiation. It is widely involved in inflammation, energy expenditure, oxidative stress, aging, neural signaling, and even circadian rhythms. Consequently, SIRT1 is able to play important roles in various diseases.

In recent years, SIRT1 has been considered a protein molecule associated with asthma. The therapeutic potential of SIRT1 activation in alleviating asthma makes this enzyme a highly attractive target for drug design. SIRT1 modulators are considered potential drugs for the treatment of asthma. However, whether SIRT1 is involved in bronchial asthma induced by stress and inflammatory responses in airway epithelial cells remains unknown. Specifically, whether changes in SIRT1 expression accompany airway epithelial cell injury, and whether SIRT1 is expressed in airway epithelial cells to regulate the pathogenesis of bronchial asthma, have not yet been reported.

SRT2104 (C₂₆H₂₄N₆O₂S₂, CAS 1093403-33-8) is the first highly selective small-molecule activator of SIRT1. Currently, research on SRT2104 has progressed to the stage of clinical translation. Unfortunately, SRT2104 is primarily administered orally or intravenously. After entering the intestine, it is absorbed by intestinal epithelial cells, leading to a first-pass effect in the liver, which affects the efficacy of SRT2104 and results in poor targeting. Therefore, it is an urgent technical problem in the field that needs to be addressed: whether SRT2104 can be directly delivered to airway epithelial cells through alternative routes of administration, and whether it can be developed as a new drug for the treatment of bronchial asthma by acting on the SIRT1 target.

Further research in the disclosure has revealed that SRT2104 is well tolerated. After being administered via nebulization and inhaled into the body, it exhibits high targeting and acts specifically on airway epithelial cells. Off-target effects and the first-pass effect in the liver are not currently observed. Moreover, the dosage required for nebulized administration is ¼ to 1/10 of that required for intravenous injection. The drug acts directly on airway epithelial cells, alleviates bronchial asthma, and takes effect rapidly.

### SUMMARY

In view of this, the disclosure is proposed.

To achieve the above purpose, the following technical solutions are adopted by the disclosure.

Specifically, a use of SRT2104 in preparation of a drug for treatment of bronchial asthma is provided, and SRT2104 enhances deacetylation activity of SIRT1 on nuclear factor kappa B subunit p65 (NF-κB p65).

In an embodiment, SRT2104 is used to reduce production of inflammatory factors induced by lipopolysaccharide (LPS) in human bronchial epithelial (HBE) cells, and the inflammatory factors are interleukin 25 (IL-25), thymic stromal lymphopoietin (TSLP), interleukin 33 (IL-33) and tumor necrosis factor alpha (TNF-α).

In an embodiment, intervention of the SRT2104 is capable of reducing infiltration of inflammatory cells in bronchoalveolar lavage fluid (BALF) of asthmatic mice and reducing inflammation score of lung tissue.

In an embodiment, SRT2104 is capable of reducing secretion of mucus in airways in asthmatic mice.

In an embodiment, SRT2104 is capable of increasing an expression level of the SIRT1 in lung tissue of asthmatic mice.

Through the above technical solutions, compared with the related art, the role and anti-inflammatory mechanism of SRT2104 in inhibiting airway epithelial inflammation by activating SIRT1 are clarified by the disclosure. In the disclosure, HBE and SIRT1-knockout HBE cell lines are used, inflammatory responses are induced, and SRT2104 is applied. The levels of inflammatory factors, the activation degree of SIRT1, and the acetylation level of NF-κB are detected. In addition, an asthma model is successfully constructed with wild-type mice, and SRT2104 is administered via nebulization. The levels of inflammatory factors in BALF, the levels of airway epithelium-specific inflammatory factors, the infiltration of inflammatory cells in lung tissue, the secretion of airway mucus, and the expression level of SIRT1 in lung tissue are observed. Through these experiments, the activation effect of SRT2104 on airway epithelial SIRT1 under asthma conditions and its alleviating effect on asthma airway inflammation are comprehensively evaluated. The effectiveness of SRT2104 in activating airway epithelial SIRT1 and controlling asthma airway inflammation under asthma conditions is confirmed.

Moreover, the disclosure adopts direct administration via nebulization, which is different from current oral or intravenous injection methods. In this situation, the dosage is greatly reduced, and asthma symptoms are rapidly relieved in a short time with almost no side effects. Furthermore, SRT2104 targets the SIRT1 protein in airway epithelial cells, and the therapeutic effect on bronchial asthma is achieved by regulating the expression level of the SIRT1 protein. A foundation is laid for the development of targeted drugs for the clinical treatment of bronchial asthma.

### BRIEF DESCRIPTION OF DRAWINGS

To illustrate embodiments of the disclosure or technical solutions in the related art more clearly, the accompanying drawings used in the embodiments or description of the related art are briefly introduced below. Apparently, the accompanying drawings in the following description are only the embodiments of the disclosure. For those skilled in the art, other drawings can also be obtained from the provided drawings without creative effort.

FIG. 1 illustrates a knockout effect of a SIRT1 gene in HBE.

FIGS. 2A-2B illustrate expression levels of a SIRT1 protein in HBE under different concentrations of SRT2104. Specifically, FIG. 2A illustrates the expression levels of SIRT1 protein under different concentrations of SRT2104 detected by Western blot. FIG. 2B illustrates the expression levels of SIRT1 protein statistically analyzed by protein gray scale analysis with Tubulin as an internal reference. Data are expressed as mean±standard deviation. NS: P>0.05; ****: P<0.0001.

FIG. 3 illustrates survival rates of HBE under different concentrations of LPS.

FIGS. 4A-4C illustrate expression levels of IL-33, IL-25, and TSLP in HBE under different concentrations of LPS.

FIGS. 5A-5B illustrate that SRT2104 effectively activates SIRT1 in LPS-induced inflammatory responses.

FIGS. 6A-6C illustrate that SRT2104 reduces production of LPS-induced inflammatory factors in HBE.

FIGS. 7A-7D illustrate that SRT2104 has no alleviating effect on the inflammatory response in SIRT1-knockout HBE.

FIG. 8 illustrates that SRT2104 has no effect on the deacetylation activity of NF-κB p65 in SIRT1-knockout HBE.

FIGS. 9A-9C illustrate detection of inflammatory factors in plasma.

FIGS. 10A-10E illustrate that intervention of SRT2104 reduces infiltration of inflammatory cells in BALF of asthmatic mice.

FIGS. 11A-11B illustrate hematoxylin and eosin (HE) staining and inflammation scoring of lung tissue.

FIGS. 12A-12B illustrate periodic acid-Schiff (PAS) staining and inflammation scoring of lung tissue.

FIG. 13 illustrates expression of SIRT1 around the airway in mice, as observed by immunohistochemistry (IHC).

### DETAILED DESCRIPTION OF EMBODIMENTS

Technical solutions in embodiments of the disclosure are clearly and completely described below in conjunction with the accompanying drawings. It should be understood that the described embodiments are only a part of the embodiments of the disclosure, rather than all of them. Based on the embodiments of the disclosure, all other embodiments obtained by those skilled in the art without creative effort fall within the protection scope of the disclosure.

Experimental materials: HBE cells are provided by the Maternal and Child Health Research Institute of Shunde Women and Children's Hospital, Affiliated to Guangdong Medical University, China. Female C57BL/6 mice of specific pathogen-free (SPF) grade are used. The mice are aged 6-8 weeks, with a body weight of 20±4 grams (g), and are purchased from Guangdong Medical Laboratory Animal Center, China.

The main equipment is shown in Table 1.

TABLE 1

| | |
|---|---|
| Adjustable micropipette | Eppendorf, Germany |
| Electronic analytical balance | SHIMADZU, Japan |
| Real-time quantitative polymerase | Thermo Fisher Scientific, USA |
| chain reaction (PCR) system | |
| PCR amplifier | Bio-Rad, USA |
| High-speed refrigerated | Thermo Fisher Scientific, USA |
| microcentrifuge | |
| Carbon dioxide (CO₂) cell culture | Thermo Fisher Scientific, USA |
| incubator | |
| Fluorescence inverted microscope | Carl Zeiss, Germany |
| Constant temperature water bath | Qun'an Laboratory Instruments, |
| | China |
| Vortex mixer | Shanghai Huxi Industrial, China |
| Microplate reader | BioTek, USA |
| Protein electrophoresis system | Bio-Rad, USA |
| TS-1 decolorizing shaker | Qilin Medical Instruments, China |
| Chemiluminescence imaging system | Bio-Rad, USA |
| (i.e, Western blot imaging system) | |
| Automated cell counter | DeNovix Inc., USA |

The main reagents are shown in Table 2

TABLE 2

| | |
|---|---|
| Anti-SIRT1 antibody | Abcam, USA |
| SRT2104 | GLPBIO, USA |
| Dimethyl sulfoxide (DMSO) | Thermo Fisher Scientific, |
| | USA |
| Antibody dilution buffer | Zhongshan Golden Bridge |
| | Biotechnology, China |
| Phosphate buffered saline (PBS) powder | Biosharp, China |
| Bicinchoninic acid (BCA) protein | Beyotime Biotechnology, |
| assay kit | China |
| Mouse enzyme-linked immunosorbent | Wuhan Immune Biology, |
| assay (ELISA) kit | China |
| Premium fetal bovine serum | Gbico, Australia |
| Lipofectamine ™ 3000 transfection | Thermo Fisher Scientific, |
| reagent | USA |
| 0.25% Trypsin-Ethylenediaminetetraacetic | Gibco, USA |
| acid (EDTA) solution | |
| Ovalbumin | Sigma-Aldrich, Germany |
| Goat serum | Beyotime Biotechnology, |
| | China |
| Anti-NF-κB antibody | Synbio Technologies, |
| | China |
| Sodium dodecyl sulfate- | Beyotime Biotechnology, |
| polyacrylamide gel | China |
| electrophoresis (SDS-PAGE) | |
| gel preparation kit | |
| Reverse transcription kit | NovoProtein, China |

reagent

| | |
|---|---|
| Trizol ™ reagent | Invitrogen, USA |
| qPCR master mix | Tsingke Biotechnology, |
| | China |
| Ready-to-use mouse anesthetic | Aibei Biology, China |
| EDTA antigen retrieval solution | Servicebio, China |
| 3,3′-Diaminobenzidine (DAB) chromogen | Servicebio, China |
| kit for immunohistochemistry | |

### Preparation of Main Reagents

(1) Tris-buffered saline with TWEEN 20 (TBST): 50 milliliters (mL) of 20× Tris-buffered saline (TBS) is combined with 949 mL of ultrapure water, and then 1 mL of polysorbate-20 (TWEEN-20) solution is added. The buffer is stirred thoroughly until the liquid becomes clear, and is stored at room temperature.

(2) PBS buffer: One packet of PBS powder is dissolved in 2000 mL of purified water by thorough stirring. After the powder is completely dissolved, the solution is sterilized by steam for 30 minutes and is stored in a refrigerator at 4° C.

(3) 5% skim milk blocking solution: 5 milligrams (mg) of skim milk powder is weighed and added to 100 mL of TBST solution. The mixture is fully homogenized and is stored at −20° C.

(4) Goat serum blocking solution: 1 mL of 10% goat serum is mixed with 9 mL of PBS. The mixture is homogenized and then incubated in a 55° C. water bath for 26 minutes. It is subsequently stored at −20° C.

(5) Ovalbumin (OVA) suspension for intraperitoneal injection: 480 micrograms (μg) of OVA is added to 2.4 mL of physiological saline, followed by the addition of 24 mg of aluminum hydroxide (Al(OH)₃). The mixture is thoroughly homogenized and is used immediately after preparation.

(6) 5% OVA nebulization solution: 5 g of OVA (ovalbumin from chicken) is dissolved in 100 mL of 0.9% physiological saline by stirring until homogenized, and is stored at 4° C.

(7) SRT2104 nebulization solution: 4 mg of SRT2104 is dissolved in 1 mL of DMSO and thoroughly mixed. Then, 4 mL of polyethylene glycol 300 (PEG 300) and 1 mL of TWEEN is added sequentially. Finally, physiological saline is added to bring the total volume to 50 mL. The solution is incubated in a 37° C. water bath for 10 minutes and is stored at 4° C.

### Embodiment 1

### Culture of HBE Cells

### (1) Thawing of HBE Cells

\textcircled{1} Dulbecco's modified eagle medium (DMEM) complete medium is removed from 4° C. and is allowed to equilibrate at room temperature for 30 minutes.

\textcircled{2} The water bath is turned on and is set to 37° C.

\textcircled{3} The cryopreserved cells are immediately placed into the water bath and are thawed for 3 minutes.

\textcircled{4} The cells are transferred into a centrifuge tube that has already been filled with complete medium, and are centrifuged at 850 revolutions per minute (rpm) for 4 minutes.

\textcircled{5} The supernatant in the centrifuge tube is discarded, and complete medium is added. The cell pellet at the bottom of the tube is gently resuspended by pipetting.

\textcircled{6} The resuspended cells are transferred into a culture dish, are gently mixed by slow shaking, and after 15 minutes of rest, the dish is carefully placed into a cell incubator (37° C., 5% CO₂, 95% humidity) for culturing.

### (2) Passage of HBE Cells

\textcircled{1} DMEM complete medium, 0.25% EDTA trypsin, and 1×PBS are removed from 4° C. and are allowed to equilibrate at room temperature for 30 minutes.

\textcircled{2} The culture medium in the dish is discarded, and the cells are washed twice with 1×PBS.

\textcircled{3} A small amount of 0.25% EDTA trypsin is added to the dish and is incubated in the cell incubator for 3 minutes for digestion.

\textcircled{4} When the cells begin to flow like quicksand, it is indicated that digestion is complete, and complete medium is immediately added to terminate digestion.

\textcircled{5} The medium is gently pipetted multiple times to ensure complete detachment of cells from the dish.

\textcircled{6} The cell suspension is transferred into a centrifuge tube and is centrifuged at 850 rpm for 4 minutes.

\textcircled{7} Steps (1)-5-6 are followed.

### (3) Cryopreservation of HBE Cells

\textcircled{1} Under microscopic observation, cells are cryopreserved when they reach 90% confluence.

\textcircled{2} Steps (2)-1-6 are followed.

\textcircled{1} The supernatant in the centrifuge tube is discarded, and cell cryopreservation medium is added. The cells are repeatedly pipetted to resuspend them in the cryopreservation medium. The cells are transferred into cryovials at a density of 1×10⁶ viable cells/mL. The vials are sealed, labeled with cell information, and are stored in a −80° C. freezer. For long-term storage, the vials are transferred to a liquid nitrogen tank the next day.

### Embodiment 2 Construction of HBE Cell Line with SIRT1 Gene Knockout

### (1) Determination of Puromycin Selection Concentration

\textcircled{1} HBE cells are seeded uniformly into a 12-well culture plate at a density of 2×10⁵ cells per well and are incubated overnight in a cell incubator.

\textcircled{2} The culture medium in the plate is discarded, and the cells are washed twice with 1×PBS.

\textcircled{3} Selection medium containing different concentrations of puromycin is added, and the plate is placed into the cell incubator for culturing. (The concentrations of puromycin are: 0, 0.3, 0.5, 1, 2, 5, 10, 15 micrograms per liter abbreviated as μg/mL.)

\textcircled{4} Cell growth is observed after 48 hours, and fresh selection medium is replaced.

\textcircled{5} Cell viability is observed and recorded daily. The lowest concentration that effectively kills all cells after 72 hours of culture is selected as the puromycin selection concentration.

### (2) Lentiviral Infection of HBE Cells

\textcircled{1} 293T packaging cells are thawed and are seeded uniformly into a 6-well culture plate at a density of 1×10⁶ cells per well, and are incubated overnight in a cell incubator.

\textcircled{2} When the cell confluence reaches 70%, the recombinant plasmid PiggyBac transposon-SIRT1 gene-single guide RNA (PB-SIRT1-sgRNA) is transfected into the 293T cells.

\textcircled{3} After 48 hours of transfection, the cell supernatant is collected. The collected supernatant is concentrated according to the instructions of the lentivirus concentration kit.

\textcircled{4} HBE cells are thawed and are seeded uniformly into a 12-well culture plate at a density of 1×10⁵ cells per well, and are cultured in the cell incubator.

\textcircled{5} When the cell confluence reaches 60%, the medium is replaced with fresh medium. The concentrated virus is added to infect the HBE cells, along with 1 μL/mL polybrene to enhance infection efficiency, and the plate is placed into the cell incubator for culturing.

\textcircled{6} After 8 hours of infection, the medium is discarded, the cells are washed twice with 1×PBS, and fresh medium is added.

\textcircled{7} After 72 hours of infection, puromycin (0.5 μg/mL) is added for selection over 3 days. When sufficient cells are obtained, a portion is used to assess the expression level of SIRT1.

### (3) Western Blot Analysis of SIRT1 Protein Expression

\textcircled{1} The medium is discarded, the cells are washed three times with 1×PBS, protease inhibitor is added, and the cells are placed on ice for 40 minutes. The cells are then scraped, transferred into a centrifuge tube, centrifuged at 12,000 rpm and 4° C. for 15 minutes, and the supernatant is collected.

\textcircled{2} Protein concentration is determined, the samples are aliquoted, and are stored at −80° C.

\textcircled{3} Separating gel and stacking gel are prepared according to the instructions, and are allowed to set at room temperature for 30 minutes until fully solidified.

\textcircled{4} Protein samples and loading buffer are mixed at a 4:1 ratio, heated in boiling water for 13 minutes to denature the proteins, and cooled at room temperature for 10 minutes.

\textcircled{5} Samples are loaded into the gel wells. Electrophoresis is performed at a constant voltage of 80 volts (V) for 30 minutes. When the bromophenol blue dye reaches the interface between the stacking and separating gels, the voltage is increased to 160 V. Electrophoresis is stopped when the dye reaches the bottom of the separating gel.

\textcircled{6} A polyvinylidene fluoride (PVDF) membrane is cut to the size of the gel and is soaked in methanol for 2 minutes. The gel is removed from the glass plates, and a transfer sandwich is assembled in the order: sponge+PVDF membrane+gel+sponge. This assembly is placed into a rapid transfer apparatus and transferred for 10 minutes.

\textcircled{7} The PVDF membrane is removed, washed twice with TBST, placed into 5% skim milk blocking solution, and blocked at low speed on a shaker for 50 minutes. The membrane is then washed three times with TBST.

\textcircled{8} Primary antibody dilution for SIRT1 is prepared at a 1:900 ratio using antibody dilution buffer. The diluted primary antibody is added, and the membrane is incubated overnight on a shaker at 4° C.

\textcircled{9} The primary antibody solution is recovered, the membrane is washed five times with TBST (10 minutes each), secondary antibody diluted in 1% bovine serum albumin (BSA) solution is added, and the membrane is incubated at low speed on a shaker for 50 minutes. The secondary antibody is then recovered, and the membrane is washed five times with TBST (8 minutes each).

\textcircled{10} Enhanced chemiluminescence (ECL) detection solution (Solution A: Solution B=1:1) is prepared in the dark, mixed well, and applied to the membrane surface. After 1 minute of incubation, the membrane is imaged using a gel imaging system. Tubulin is used as the internal reference. The relative expression level of the target protein is calculated using ImageJ software and subjected to statistical analysis.

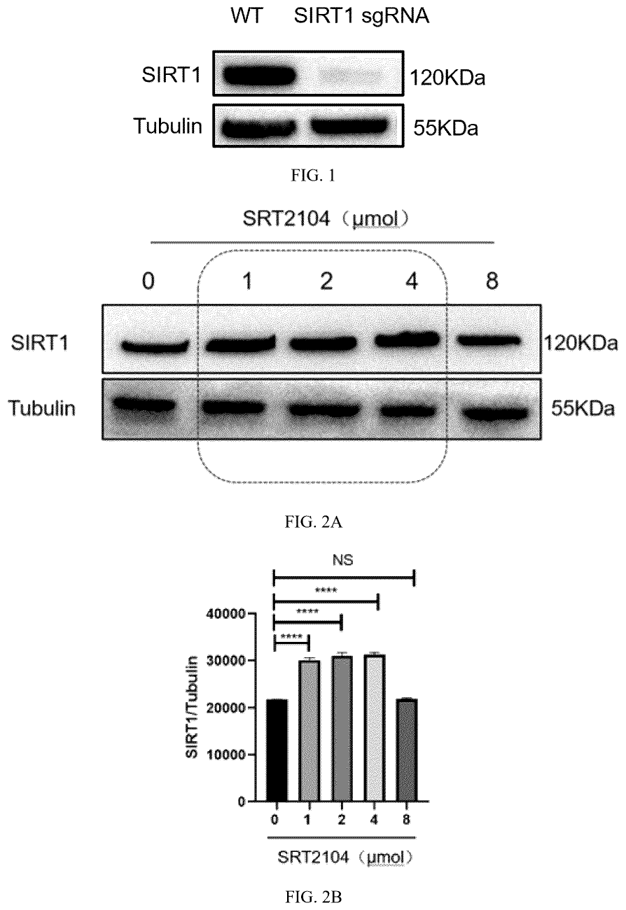

The results show that, as shown in FIG. 1, compared with wild-type HBE cells, SIRT1 protein expression is absent in the knockout group, indicating that an HBE cell line with SIRT1 gene knockout has been successfully generated.

### Embodiment 3 Western Blot Detection of SIRT1 Protein Expression Levels Under Different Concentrations of SRT2104

(1) SRT2104 at concentrations of 1 micromole per liter (μmol/L), 2 μmol/L, 4 μmol/L, and 8 μmol/L is prepared using DMSO as the solvent.

(2) HBE cells are thawed and seeded uniformly into a 6-well culture plate at a density of 1×10⁶ cells per well, and are incubated overnight in a cell incubator.

(3) When the cell confluence reaches 80%, the medium is replaced with fresh medium, and different pre-prepared concentrations of SRT2104 are added to each well. The plate is placed into the cell incubator for culturing.

(4) After 24 hours of culture, the medium is discarded, the cells are washed twice with 1×PBS, protease inhibitor is added, and the cells are placed on ice for 40 minutes. The cells are then collected using a cell scraper, transferred into a centrifuge tube, centrifuged at 12,000 rpm and 4° C. for 15 minutes, and the supernatant is collected.

(5) Steps (3)-\textcircled{2} to \textcircled{10} of the embodiment 3 are followed.

The results show that, compared with the blank control group, SIRT1 protein expression is significantly upregulated when treated with 1 μmol/L, 2 μmol/L, and 4 μmol/L of SRT2104 (P<0.0001), and the difference is statistically significant (FIG. 2A-2B). This indicates that SIRT1 is activated in HBE cells treated with 1 μmol/L, 2 μmol/L, and 4 μmol/L of SRT2104. Therefore, 1 μmol/L, 2 μmol/L, and 4 μmol/L of SRT2104 are selected for subsequent experiments.

### Embodiment 4 LPS-Induced Inflammation in HBE Cells

(1) Cell Counting Kit-8 (CCK-8) Assay to Detect the Viability of HBE Cells under Different Concentrations of LPS

\textcircled{1} LPS solutions at concentrations of 2.5 μg/mL, 5 μg/mL, 10 μg/mL, 15 μg/mL, and 20 μg/mL are prepared using sterile distilled water as the solvent.

\textcircled{2} HBE cells are thawed. Five experimental groups are set according to the prepared LPS concentrations, with six replicate wells per group. A blank control group is also set. PBS buffer is added to the outermost wells of a 96-well plate to reduce medium evaporation. Cells are seeded uniformly at a density of 2×10³ cells per well and are incubated overnight in a cell incubator.

\textcircled{3} When the cell confluence reaches 85%, the medium is discarded, and the five experimental groups are treated with culture medium containing different concentrations of LPS, while the blank control group is treated with an equal volume of culture medium. The plate is incubated in the cell incubator for 24 hours.

\textcircled{4} Fresh medium is added, and 10 μL of CCK-8 solution is added to each well, taking care to avoid bubble formation. The 96-well plate is then placed into the cell incubator for 1 hour.

\textcircled{5} The 96-well plate is removed, protected from light, and the optical density (OD) value at 450 nanometers (nm) is measured using a microplate reader. Cell viability is calculated as: [(OD of experimental well−OD of blank well)/(OD of control well−OD of blank well)]×100%. Data are analyzed and a curve is plotted.

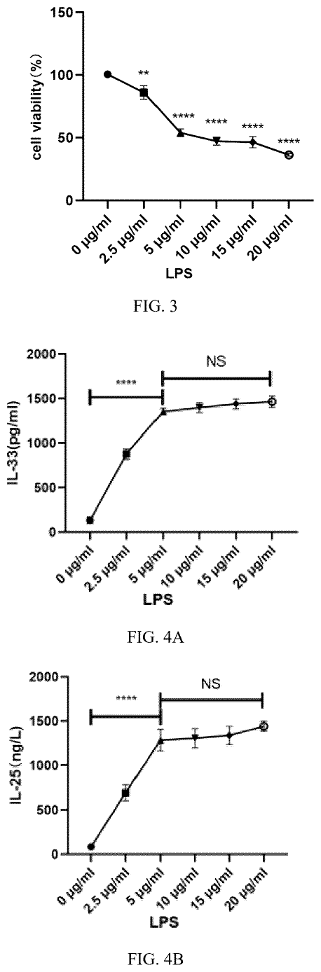

The results show that the HBE cell viability is (86.2±5.4) % in the 2.5 μg/mL LPS group, (54.0±3.5) % in the 5 μg/mL LPS group, (47.3±3.5) % in the 10 μg/mL LPS group, (46.6±4.9) % in the 15 μg/mL LPS group, and (36.5±1.2) % in the 20 μg/mL LPS group. This indicates that LPS affects HBE cell viability, and viability gradually decreases as the LPS concentration increases (FIG. 3).

(2) ELISA Detection of Inflammatory Factor Expression Levels in HBE Cells under Different Concentrations of LPS

\textcircled{1} HBE cells are thawed. Five experimental groups are set according to the prepared LPS concentrations, with three replicates per group. A blank control group is also set. Cells are seeded uniformly into a 6-well culture plate at a density of 1×10⁶ cells per well and are cultured in the cell incubator.

\textcircled{2} When the cell confluence reaches 80%, the medium is discarded, the cells are washed twice with 1×PBS, and the five experimental groups are treated with culture medium containing different concentrations of LPS, while the blank control group is treated with an equal volume of culture medium. The plate is incubated in the cell incubator for 24 hours.

\textcircled{3} The cell supernatant is collected and centrifuged at 3000 rpm for 15 minutes to remove particles and aggregates.

\textcircled{4} The ELISA kit is removed from 4° C. and is equilibrated at room temperature for 30 minutes.

\textcircled{5} The required strips are removed from the aluminum foil pouch of the kit. Standard wells and sample wells are set. 50 μL of different concentrations of standard is added to the standard wells. 40 μL of sample diluent is added to the sample wells, followed by 10 μL of the sample to be tested. The blank wells are left empty.

\textcircled{6} Except for the blank wells, 100 μL of horseradish peroxidase (HRP)-labeled detection antibody is added to each standard and sample well. The wells are sealed with a plate sealer and incubated at 37° C. for 1 hour.

\textcircled{7} The sealer is carefully removed, the liquid is discarded, and the wells are blotted dry on absorbent paper. Each well is filled with wash buffer, allowed to stand for 60 seconds, then the wash buffer is discarded and the wells are blotted dry. This washing step is repeated six times.

\textcircled{8} 50 μL of substrate solution A and 50 μL of substrate solution B are added to each well. The plate is protected from light and incubated at 37° C. for 20 minutes.

\textcircled{9} 50 μL of stop solution is added to each well, and the OD value at 450 nm is measured using a microplate reader. The concentration of inflammatory factors is calculated based on the standard curve, and a curve is plotted.

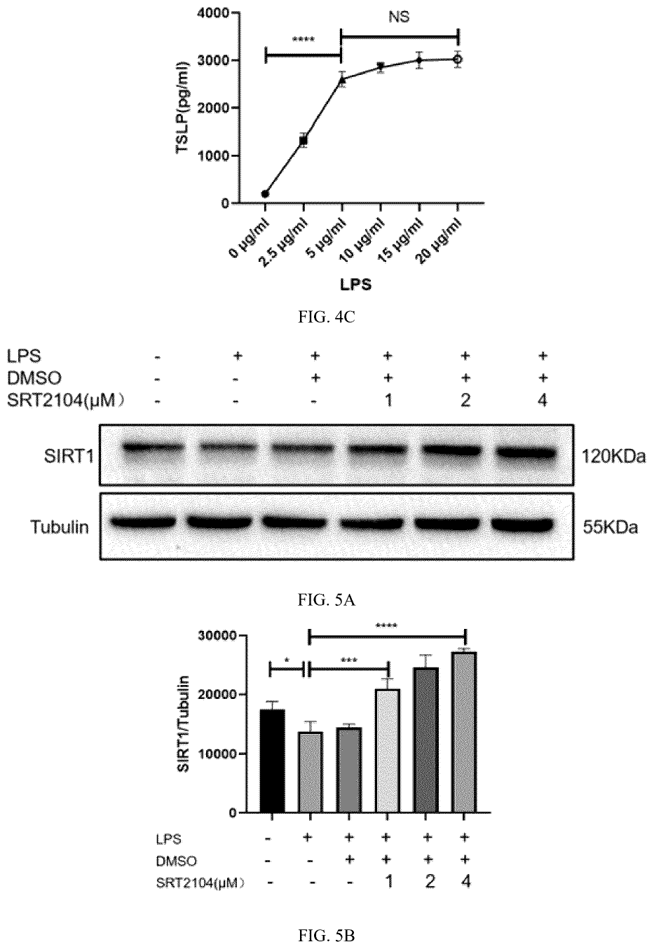

The results show that, compared with the blank control group, the expression levels of inflammatory factors IL-33, IL-25, and TSLP in HBE cells gradually increase under different concentrations of LPS. In the 5 μg/mL LPS group, the expression levels of IL-33, IL-25, and TSLP are significantly increased (P<0.0001), and the differences are statistically significant (FIGS. 4A-4C). Therefore, 5 μg/mL LPS is selected as the induction concentration for subsequent experiments.

### Embodiment 5 Effects of SRT2104 on LPS-Induced HBE Cells and SIRT1 Gene-Knockout HBE Cells

### (1) Cell Grouping

Cells are processed according to the Table 3.

TABLE 3
Cell Grouping

| | |
|---|---|
| HBE | HBE-SIRT1 sgRNA |
| HBE blank control group | HBE-SIRT1 sgRNA blank control |
| | group |
| LPS treatment group | LPS treatment group |
| LPS + solvent (DMSO) group | LPS + solvent (DMSO) group |
| LPS + 1 μmol/L SRT2104 group | LPS + 1 μmol/L SRT2104 group |
| LPS + 2 μmol/L SRT2104 group | LPS + 2 μmol/L SRT2104 group |
| LPS + 4 μmol/L SRT2104 group | LPS + 4 μmol/L SRT2104 group |

Figure Caption: HBE-SIRT1 sgRNA: airway epithelial cells with SIRT1 gene knockout.

(2) Western Blot Detection of the Expression Levels of Related Proteins in LPS-Induced HBE Cells and SIRT1 Gene-Knockout HBE Cells after SRT2104 Treatment

\textcircled{1} Culture medium containing LPS (5 μg/mL) is added according to the grouping and is used to induce the cells for 24 hours. Then, culture medium containing different concentrations of SRT2104 is added, and the cells are cultured in the cell incubator for 24 hours.

\textcircled{2} Steps (3)-1 to (7) of the embodiment 2 are followed.

\textcircled{3} Primary antibody dilutions for SIRT1, NF-κB p65, and Ac-NF-κB p65 are prepared separately using antibody dilution buffer. The diluted primary antibodies are added, and the membranes are incubated overnight on a shaker at 4° C.

\textcircled{4} Steps (3)-\textcircled{9} to \textcircled{10} of the embodiment 2 are followed.

The results show that, compared with the blank control group, SIRT1 protein expression is significantly downregulated in the LPS group (P<0.05), and the difference is statistically significant. In the high-, medium-, and low-dose SRT2104 groups, SIRT1 protein expression is gradually upregulated (P<0.001), and the differences are statistically significant (FIGS. 5A-5B). This indicates that SRT2104 can effectively activate SIRT1 in LPS-induced inflammatory responses, and this effect is dose-dependent.

(3) ELISA Detection of the Effects of SRT2104 on Related Inflammatory Factors in LPS-Induced HBE Cells and SIRT1 Gene-Knockout HBE Cells

\textcircled{1} HBE cells and SIRT1 gene-knockout HBE cells are cultured. When the cell confluence reaches 60%, the medium is discarded, the cells are washed three times with 1×PBS, and culture medium containing different concentrations of SRT2104 is added according to the grouping and is used to culture the cells for 24 hours. The medium is then replaced, and culture medium containing LPS (5 μg/mL) is added. The cells are cultured in the cell incubator for 24 hours.

\textcircled{2} Steps (2)-\textcircled{3} to \textcircled{9} of the embodiment 4 are followed.

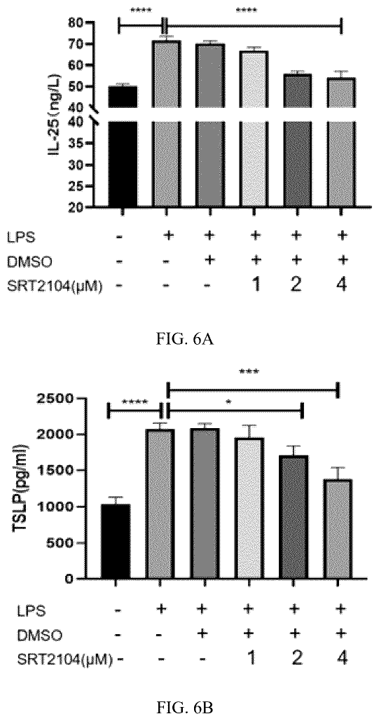

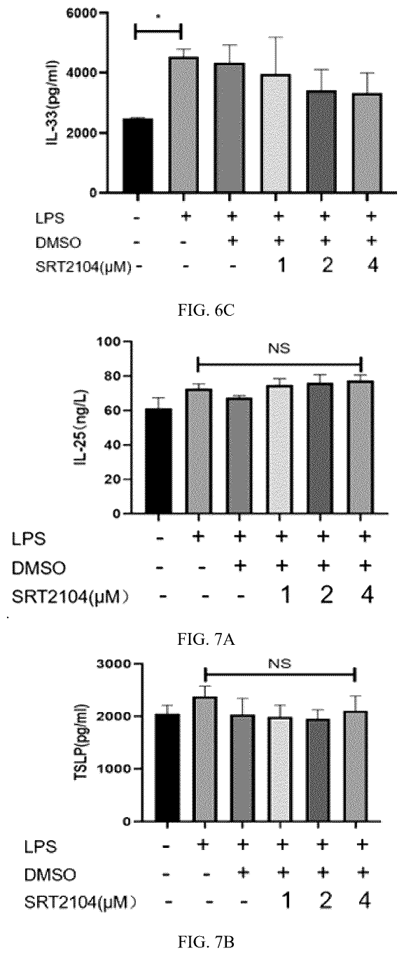

The results show that, for the HBE cell line, compared with the blank control group, the levels of IL-25, IL-33, and TSLP are significantly increased in the LPS group (P<0.05), and the differences are statistically significant. In the SRT2104 treatment groups, the levels of IL-25, IL-33, and TSLP show a decreasing trend. Among them, the levels of IL-25 and TSLP in the SRT2104 treatment groups are significantly decreased (P<0.001), and the differences are statistically significant (FIGS. 6A-6C).

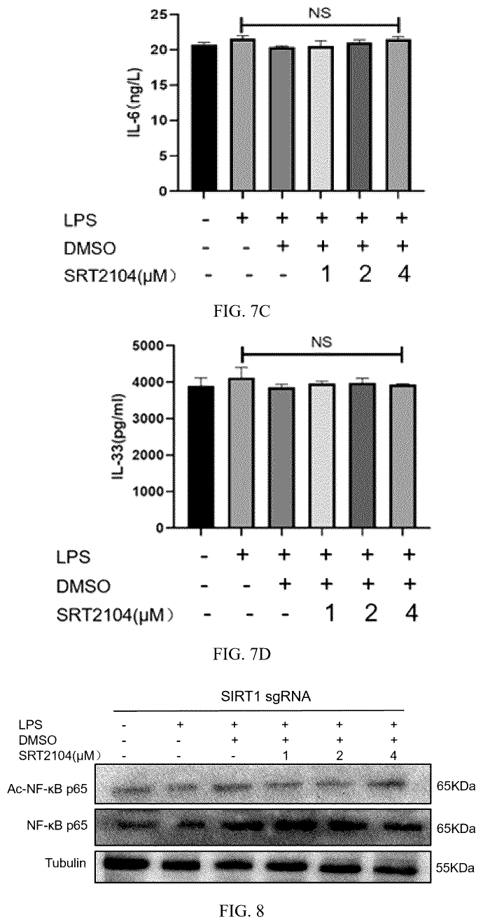

For the SIRT1 gene-knockout cell line, the results show that there are no significant differences in the levels of IL-25, TSLP, IL-6, and IL-33 between the high-, medium-, and low-dose SRT2104 groups and the LPS group (P>0.05) (FIGS. 7A-7D). This indicates that SRT2104 has no alleviating effect on the inflammatory response in HBE cells with SIRT1 gene knockout.

Meanwhile, the expression levels of NF-κB p65 and Ac-NF-κB p65 proteins are detected by Western blot. The results show that, compared with the knockout control group and the LPS group, there are no significant differences in the level of Ac-NF-κB p65 protein in the high-, medium-, and low-dose SRT2104 groups. This indicates that SRT2104 has no effect on the deacetylation activity of NF-κB p65 in HBE cells with SIRT1 gene knockout (FIG. 8).

### Embodiment 6 Animal Experiment Grouping and Establishment of the Asthma Model

### (1) Animal Grouping

\textcircled{1} Animal selection: SPF-grade C57BL/6 wild-type mice, female, 6 weeks old, with a body weight of approximately 20 g.

\textcircled{2} Experimental grouping: 24 mice are randomly divided into four groups: PBS group, OVA group, OVA+solvent (DMSO) group, and OVA+SRT2104 group, with 6 mice in each group.

### (2) Establishment of the Asthma Model

\textcircled{1} Sensitization is performed three times at 7-day intervals over 14 days by intraperitoneal injection of 40 μg OVA plus 2 mg Al(OH)₃.

\textcircled{2} From day 18 to day 20, the mice are placed into a nebulization chamber and are nebulized with PBS, DMSO, or SRT2104 for three consecutive days according to the grouping.

\textcircled{3} From day 21 to day 23, the mice are placed into a nebulization chamber and are nebulized with 5% OVA (prepared in physiological saline) for three consecutive days to induce asthma.

\textcircled{4} On day 24, the mice are euthanized, and specimens are collected.

### Embodiment 7 Detection of Inflammatory Factors in Plasma

(1) Blood Collection from the Eye

\textcircled{1} The mouse is first anesthetized. After sufficient anesthesia is confirmed, the mouse is firmly held with one hand, and the whiskers are trimmed to prevent blood contamination.

\textcircled{2} The skin around the eye on the blood collection side is gently pressed to cause the eyeball to become congested and protrude.

\textcircled{3} The eyeball is quickly removed using curved forceps, and the blood flowing from the orbit is collected. Simultaneously, the area over the mouse's heart is gently pressed with the left hand to accelerate cardiac pumping.

\textcircled{4} The collected blood is centrifuged at 2500 rpm and 4° C. for 15 minutes, and the plasma supernatant is collected into a new tube.

### (2) ELISA Detection of Inflammatory Factor Expression Levels in Plasma

\textcircled{1} Steps (2)-\textcircled{4} to \textcircled{9} of the embodiment 4 are followed.

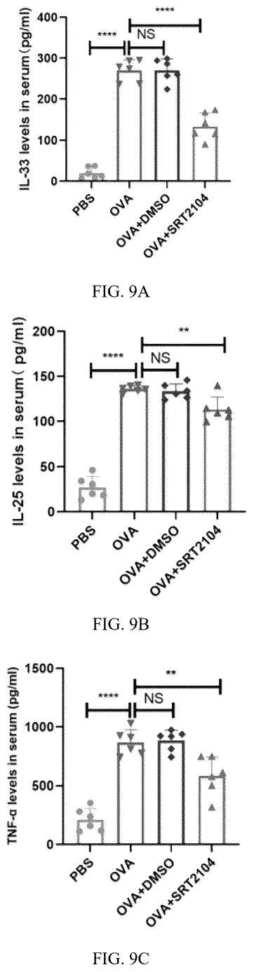

In this process, 24 C57BL/6 wild-type mice are randomly divided into four groups: PBS group, OVA group, OVA+solvent (DMSO) group, and OVA+SRT2104 group, with 6 mice per group. The asthmatic mice in the OVA+SRT2104 group are treated with 15 milligrams per kilogram (mg/kg) SRT2104 via nebulization. Twenty-four hours after the final OVA challenge, blood is collected from the eyes, and the expression levels of IL-33, IL-25, and TNF-α in the plasma of each group are detected by ELISA. The results show that, compared with the PBS group, the expression levels of IL-33, IL-25, and TNF-α are significantly increased in the OVA-induced group (P<0.0001), and the differences are statistically significant. No significant differences in the expression levels of IL-33, IL-25, and TNF-α are observed between the OVA group and the OVA+DMSO group (P>0.05). However, in the OVA+SRT2104 group, the expression levels of IL-33, IL-25, and TNF-α are significantly decreased compared to the OVA group (P<0.01), and the differences are statistically significant (FIGS. 9A-9C).

### Embodiment 8 Detection of Inflammatory Factors in BALF

### (1) Collection of BALF and Inflammatory Cells

\textcircled{1} After blood collection, the mouse is euthanized by cervical dislocation.

\textcircled{2} The chest is opened, the mediastinum is separated, and 1 mL of physiological saline is drawn into a syringe and slowly injected into the lungs via the trachea. The lavage is repeated three times, and the lavage fluid is then withdrawn.

\textcircled{3} The collected lavage fluid is centrifuged at 2000 rpm and 4° C. for 20 minutes, and the supernatant and the precipitated cellular components are collected separately.

### (2) Inflammatory Cell Counting in BALF

\textcircled{1} 1 mL of physiological saline is added to the obtained cell pellet, and the cells are gently resuspended by pipetting to prepare a uniform cell suspension.

\textcircled{2} An automated cell counter is used to determine the total cell count for each group.

\textcircled{3} After counting, 10 μL of the cell suspension is used to prepare a smear, which is of appropriate thickness and is allowed to air-dry for 24 hours.

\textcircled{4} Wright-Giemsa composite stain is added to cover the entire smear and is allowed to stain at room temperature for 1 minute.

\textcircled{5} An equal volume of PBS (pH 6.4-6.8) is added, the slide is gently agitated to mix thoroughly with the Wright-Giemsa stain, and staining is continued at room temperature for 3 minutes.

\textcircled{6} The slide is rinsed three times with near-neutral water, dried, and examined under a microscope.

### (3) ELISA Detection of Inflammatory Factor Expression Levels in BALF

\textcircled{1} Steps (2)-\textcircled{4} to \textcircled{9} of the embodiment 4 are followed.

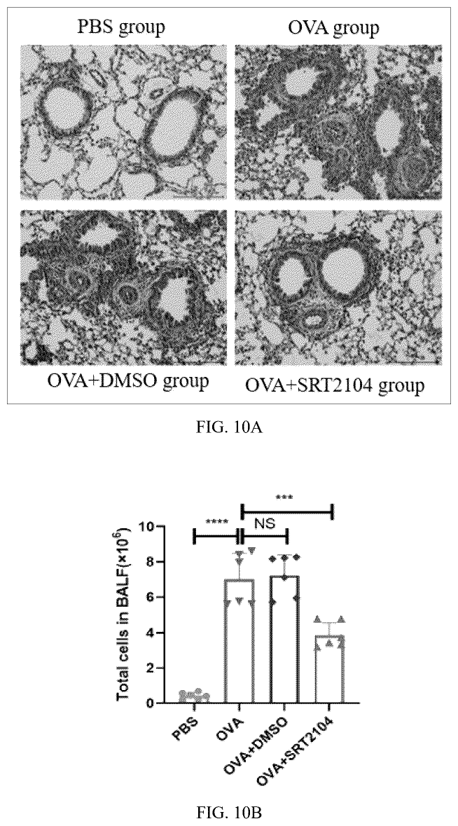

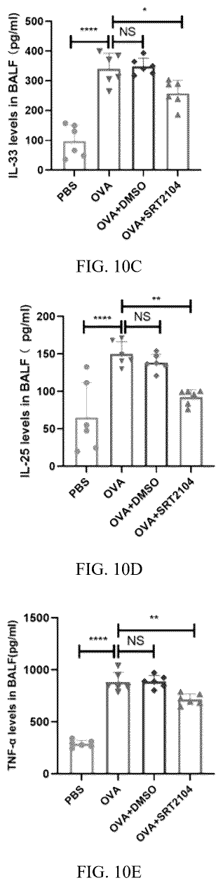

The results show that the total cell count in BALF is significantly higher in the OVA group and the OVA+DMSO group than in the PBS group (P<0.0001), and the differences are statistically significant. In the OVA+SRT2104 group, the total cell count in BALF is significantly decreased compared to the OVA and OVA+DMSO groups (P<0.001), and the difference is statistically significant. Wright-Giemsa staining results show that the total number of inflammatory cells in BALF is markedly higher in the OVA and OVA+DMSO groups than in the PBS group, while the total number of inflammatory cells in the OVA+SRT2104 group is significantly lower than in the OVA group. Detection of inflammatory factors in the BALF supernatant shows that, compared with the PBS group, the expression levels of IL-33, IL-25, and TNF-α are significantly increased in the OVA-induced group (P<0.0001), and the differences are statistically significant. No significant differences in the expression levels of IL-33, IL-25, and TNF-α are observed between the OVA group and the OVA+DMSO group (P >0.05). However, in the OVA+SRT2104 group, the expression levels of IL-33, IL-25, and TNF-α are significantly decreased compared to the OVA group (P<0.05), and the differences are statistically significant (FIGS. 10A-10E).

### Embodiment 9 Lung Tissue Histopathological Examination and Inflammation Scoring

### (1) Lung Tissue Sectioning

\textcircled{1} The left lung is excised, ensuring the integrity of the lung tissue and avoiding damage.

\textcircled{2} The excised lung tissue is placed into an appropriate fixative (4% paraformaldehyde) to prevent tissue deformation.

\textcircled{3} The tissue is subjected to dehydration through a graded ethanol series of increasing concentrations to impart rigidity.

\textcircled{4} The dehydrated specimen is thoroughly infiltrated with paraffin wax.

\textcircled{5} The specimen is embedded in paraffin wax and cut into thin sections of 5 μm thickness. All sections are oriented toward the distal airways and are used for IHC, PAS staining, and HE staining.

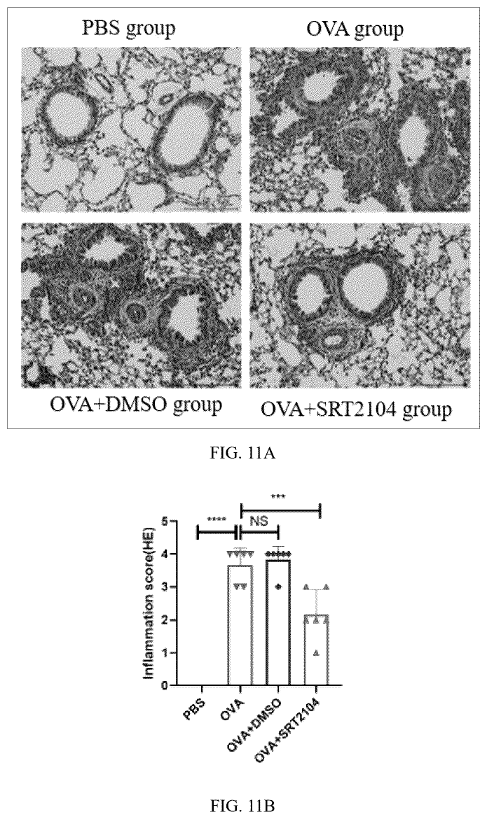

HE staining is performed to observe inflammatory cell infiltration in lung tissue. The results show that, compared with the PBS group, extensive inflammatory cell infiltration around the trachea and blood vessels is observed in the OVA group and the OVA+DMSO group. The inflammation score is estimated based on the area of inflammatory infiltration (P<0.0001), and the difference is statistically significant. In the OVA+SRT2104 group, inflammatory cell infiltration around the trachea and blood vessels is significantly reduced compared to the OVA and OVA+DMSO groups. The inflammation score is estimated based on the area of inflammatory infiltration (P<0.001), and the difference is statistically significant (FIGS. 11A-11B).

### (2) PAS Staining

\textcircled{1} The sections are placed in an oven at 60° C. for 20 minutes.

\textcircled{2} Deparaffinization: The sections are sequentially placed into xylene I for 8 minutes, xylene II for 8 minutes, absolute ethanol for 5 minutes, 95% alcohol for 5 minutes, 80% alcohol for 5 minutes, 75% alcohol for 5 minutes, and TBST for 10 minutes.

\textcircled{3} The sections are immersed in PAS staining solution for 8 minutes. After removal from the PAS solution, they are rinsed with running water for 10 minutes.

\textcircled{4} The sections are immersed in Schiff reagent for 30 minutes and rinsed with running hot water for 3 minutes.

\textcircled{5} The sections are immersed in hematoxylin reagent for 5 minutes and rinsed with running water for 5 minutes.

\textcircled{6} An appropriate amount of Bluing reagent is added to cover the sections, incubated for 1 minute, and rinsed with running water for 2 minutes.

\textcircled{7} The sections are incubated in Light green reagent for 3 minutes and then soaked in distilled water for 1 minute.

\textcircled{8} Rapid dehydration (to facilitate slide preservation): The sections are sequentially dehydrated in absolute ethanol for 5 minutes, 95% alcohol for 5 minutes, 80% alcohol for 5 minutes, 75% alcohol for 5 minutes, xylene I for 2 minutes, and xylene II for 2 minutes.

\textcircled{9} Mounting: The sections are removed from xylene, allowed to dry slightly, neutral balsam is added, a coverslip is applied, and gentle pressure is applied to allow the balsam to spread rapidly and cover the tissue. This step is performed quickly to prevent drying. Microscopic examination is performed.

\textcircled{10} PAS Staining Result Interpretation: Airway mucus secretion capacity is evaluated based on staining results: glycogen and polysaccharide substances appear purple-red, and cell nuclei appear blue. Scoring criteria: 0 points: no inflammation detected; 1 point: occasional presence of a few inflammatory cells; 2 points: one to three inflammatory cells are visible around most bronchioles or blood vessels; 3 points: four to five inflammatory cells are visible around most bronchioles or blood vessels; 4 points: more than five inflammatory cells are visible around most bronchioles or blood vessels. Three to five fields are randomly selected on each section, and a double-blind evaluation method is used. The average score for each group is taken as the final result.

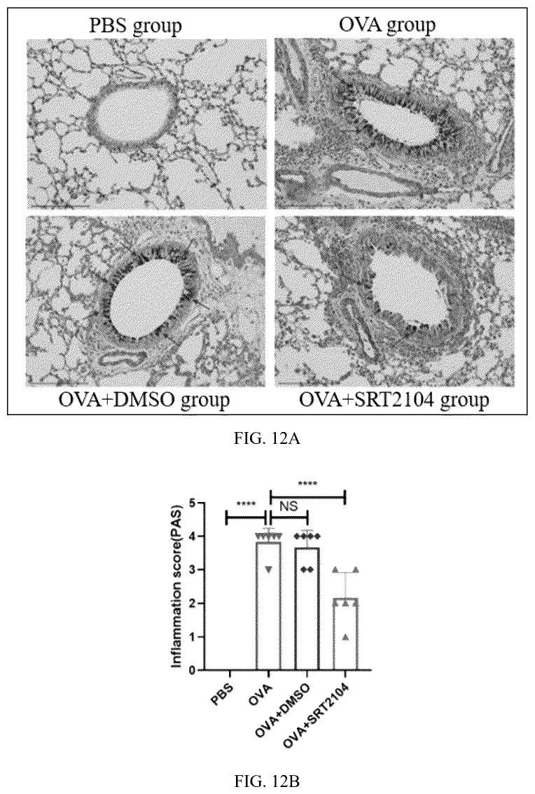

Compared with the PBS group, abundant mucus formation is observed in the cytoplasm of airway epithelial cells in the OVA group and the OVA+DMSO group. Airway mucus secretion capacity is assessed based on staining results (P<0.0001), and the difference is statistically significant. In the OVA+SRT2104 group, mucus synthesis in the cytoplasm of airway epithelial cells is significantly reduced compared to the OVA and OVA+DMSO groups. Airway mucus secretion capacity is assessed based on staining results (P<0.0001), and the difference is statistically significant (FIGS. 12A-12B).

### (3) IHC Dual-Color Staining

\textcircled{1} Steps (2)-\textcircled{1} to \textcircled{2} are followed.

\textcircled{2} Antigen retrieval: Antigen retrieval solution is prepared with deionized water. The sections are placed into the antigen retrieval solution and boiled for 20 minutes. After natural cooling, the slides are placed into PBS (pH 7.4) and washed three times on a decolorization shaker, 5 minutes each time.

\textcircled{3} Blocking endogenous peroxidase: The sections are placed into a 3% hydrogen peroxide solution and incubated at room temperature, protected from light, for 25 minutes. The slides are then placed into PBS (pH 7.4) and washed three times on a decolorization shaker, 5 minutes each time.

\textcircled{4} Blocking: 3% BSA is added into the histological circle and uniformly covers the tissue. The sections are blocked at room temperature for 30 minutes.

\textcircled{5} Primary antibody application: The blocking solution is gently removed. The primary antibody (Anti-SIRT1) is diluted at a ratio of 1:100. 100 μL of the primary antibody is added onto the section. The sections are placed flat in a humidified chamber and incubated at 4° C. overnight.

\textcircled{6} Secondary antibody application: The slides are placed into PBS (pH 7.4) and washed three times on a decolorization shaker, 5 minutes each time. After slightly drying, 100 μL of secondary antibody (HRP-labeled) is added into the circle to cover the tissue, and the sections are incubated at room temperature for 60 minutes.

\textcircled{7} DAB chromogenic reaction: The slides are placed into PBS (pH 7.4) and washed three times on a decolorization shaker, 5 minutes each time. After slightly drying, freshly prepared DAB chromogenic solution is added into the circle. The chromogenic time is controlled under a microscope, with a positive result appearing brown-yellow. The sections are rinsed with tap water to stop the reaction.

\textcircled{8} Nuclear counterstaining: The sections are counterstained with hematoxylin for approximately 3 minutes, rinsed with tap water, differentiated with hematoxylin differentiation solution for several seconds, rinsed with tap water, blued with hematoxylin bluing solution, and finally rinsed with running water.

\textcircled{9} Dehydration and mounting: The sections are sequentially dehydrated and cleared in 75% alcohol for 5 minutes, 85% alcohol for 5 minutes, absolute ethanol I for 5 minutes, absolute ethanol II for 5 minutes, and xylene I for 5 minutes. The sections are removed from xylene, allowed to dry slightly, and mounted.

\textcircled{10} Microscopic examination: Image analysis is performed under a bright-field microscope.

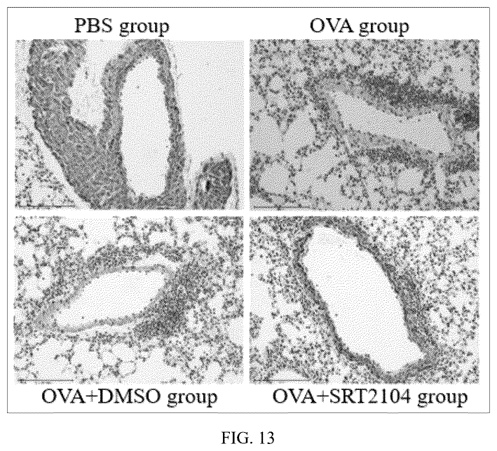

IHC staining is used to observe the expression of SIRT1 around the airways in each group of mice. The results show that SIRT1 is highly expressed in the airway epithelium of the PBS group. Compared with the PBS group, the expression of SIRT1 in the airway epithelium is significantly decreased in the OVA group and the OVA+DMSO group. In the OVA+SRT2104 group, the expression of SIRT1 in the airway epithelium is significantly increased compared to the OVA and OVA+DMSO groups. This indicates that SIRT1 is highly expressed in the airway epithelium and is downregulated during asthma, and SRT2104 can enhance the expression level of SIRT1 in the lung tissue of asthmatic mice (FIG. 13).

The various embodiments described in this specification are presented in a progressive manner, with each embodiment focusing on the aspects that differ from the other embodiments. Common and similar parts among the embodiments can be referenced mutually.

The above description of the disclosed embodiments enables those skilled in the art to implement or utilize the disclosure. Various modifications to these embodiments will be readily apparent to those skilled in the art. The general principles defined herein can be implemented in other embodiments without departing from the spirit or scope of the disclosure. Therefore, the disclosure will not be limited to the embodiments shown herein but will conform to the widest scope consistent with the principles and novel features disclosed herein.

What is claimed is:

1. A use of SRT2104 in preparation of a drug for treatment of bronchial asthma, wherein the SRT2104 is used to enhance deacetylation activity of silent information regulator 1 (SIRT1) on nuclear factor kappa B subunit p65 (NF-κB p65).

2. The use as claimed in claim 1, wherein the SRT2104 is used to reduce production of inflammatory factors induced by lipopolysaccharide (LPS) in human bronchial epithelial (HBE) cells, and the inflammatory factors comprises interleukin 25 (IL-25), thymic stromal lymphopoietin (TSLP), interleukin 33 (IL-33) and tumor necrosis factor alpha (TNF-α).

3. The use as claimed in claim 1, wherein intervention of the SRT2104 is capable of reducing infiltration of inflammatory cells in bronchoalveolar lavage fluid (BALF) of asthmatic mice and reducing inflammation score of lung tissue.

4. The use as claimed in claim 1, wherein the SRT2104 is capable of reducing secretion of mucus in airways in asthmatic mice.

5. The use as claimed in claim 1, wherein the SRT2104 is capable of increasing an expression level of the SIRT1 in lung tissue of asthmatic mice.
